(19) United States
(12) Patent Application Publication
(10) Pub. No.: US 2021/0096122 A1
(43) Pub. Date: Apr. 1, 2021

(54) METHOD FOR CONTINUOUSLY EVALUATING MECHANICAL AND MICROSTRUCTURAL PROPERTIES OF A METALLIC MATERIAL, IN PARTICULAR STEEL, IN A COLD DEFORMATION PROCESS AND RELATED APPARATUS

(71) Applicant: MARCEGAGLIA CARBON STEEL S.P.A., Ippliti (MN) (IT)

(72) Inventor: Alessandro FERRAIUOLO, Ravenna (IT)

(21) Appl. No.: 16/499,332

(22) PCT Filed: Mar. 29, 2018
(86) PCT No.: PCT/IB2018/052172

§ 371 Date: Sep. 30, 2019

(30) Foreign Application Priority Data
Mar. 31, 2017 (IT) 102017000035735

(51) Int. Cl. G01N 33/204; G01N 3/08; G01N 3/20

(52) U.S. Cl. CPC G01N 33/204; G01N 3/08; G01N 3/20; G01N 2203/0282; G01N 2203/0019; G01N 2203/0023; G01N 2203/0026; G01N 2203/0017

(57) ABSTRACT

A method is described for continuously evaluating mechanical and micro structural properties of a rolled metallic material (L) in a cold deformation process, subjected to combinations of deformation forces selected among compression forces, traction forces and bending moment applied at low deformation speed in a range comprised between 1*10⁻⁴ and 10*10⁻⁴ s⁻¹ which corresponds to laboratory static conditions and at high deformation speed in a range comprised between 0.1 and 10 s⁻¹ which corresponds to dynamic pp conditions, the method comprising the step of: —measuring characteristic parameters of the cold deformation process under dynamic conditions, comprising at least one value of temperature (T), deformation (ε) and deformation speed (\acute{ε}) of the rolled sheet (L); characterized in that it further comprises the steps of: —calculating the traction yield strength at high deformation speed (σ_{YD}) according to equation (I), being: σ_{c} a compression strength of the rolled sheet (L) when a compression force (Fc) is applied thereon; σₜ a traction strength of the rolled sheet (L) when traction forces (Tin, Tout) are applied thereon; σ_{bend} a strength due to the bending of the rolled sheet (L) when a bending moment is applied thereon; and m, n, p are a first, a second and a third parameter respectively being a function of continuously-measured operating conditions of the cold deformation process and being a function of the rolled sheet (L) in terms of chemical composition and of preceding operating conditions of a hot deformation process, in terms of hot-rolling start and end temperature, winding temperature and grain size; calculating the traction yield strength at low deformation speed (σ_{YS}) according to equation (II), being: σ_{YD} the traction yield strength at high deformation speed; f a statistical optimization factor between data measured at low deformation speed and at high deformation speed; α a first characteristic parameter of the rolled sheet (L) being a function of a chemical composition of the rolled sheet (L) and of operating conditions of a hot deformation process of the rolled sheet (L); and β a second characteristic parameter of the rolled sheet (L) being a function of the cold deformation process calculated as (III), being \acute{ε} the deformation speed, Q an activation energy of the deformation of the rolled sheet (L) evaluated through laboratory tests, R the Boltzmann constant of ideal gases, and T the temperature of the rolled sheet (L).

### FIELD OF APPLICATION

The present invention relates to a method for continuously evaluating mechanical and microstructural properties of a metallic material in a cold deformation process.

The invention also relates to an apparatus for implementing such a method in the metal engineering industry, in particular in relation to the production of steels, and the following description is made with reference to this field of application with the sole purpose of simplifying the presentation thereof.

### Prior Art

The need to qualify metallic products during the various steps of the manufacturing cycle thereof in terms of mechanical and microstructural properties is well known, in particular in the metal engineering industry.

In order to meet that need, several methods were developed for measuring these mechanical and microstructural properties directly during the manufacture of the metallic product itself, methods which proved to be an essential tool for optimizing the quality of metallic products, particularly if made of steel.

A first prior art solution to perform the measurement of the mechanical and microstructural properties of a metallic material, in particular a steel, provides a drawing of samples which are subjected to traction tests under static conditions, based on which the mechanical and microstructural properties are evaluated.

That first known solution is certainly effective, but it does not allow to have a real knowledge of the mechanical and microstructural properties of the metallic material throughout the product made of it, in particular, in the case of a sheet metal, on the whole length thereof.

The need to provide a complete picture of the mechanical and microstructural properties of the metallic materials which form metallic products has caused in the last few years a huge demand for devices being capable of providing an evaluation of those properties.

To this purpose, the currently more widespread method for measuring the mechanical and microstructural properties of a metallic material, particularly in line, i.e. during the production process thereof, is based on the evaluation of the magnetic remanence of such a metallic material during the manufacture thereof. That method can be used however only for materials having ferromagnetic properties, just by virtue of the operating principle thereof.

A method for obtaining information relating to a steel rolled sheet along the whole length thereof by using a Skin Pass Mill at the end of the continuous annealing or the galvanizing line or of the pickling line and other continuous line is known from the U.S. Pat. No. 8,296,081 granted on 23 Oct. 2012 in the name of Goto et al. (Nippon Steel Corporation). In particular, the steel rolled sheet is passed in the roll system of the Skin Pass Mill where the load, strength and lengthening values are continuously detected, correlated then with the mechanical properties of the rolled sheet.

The German patent application published with No. DE 10 2012 020444 on 24 Apr. 2014 in the name of VDEH Betriebsforschungsinstitute GmbH is also known, which describes a method for measuring the yield strength of a steel sheet by using a system of tensioning and curving rolls to apply to the sheet a longitudinal strength or pull (with respect to a displacement direction thereof) and a bending moment.

The technical problem of the present invention is to provide a method for continuously evaluating mechanical and microstructural properties of a metallic material, in particular a steel, rolled in a cold deformation process in the shape of a sheet metal or a strip, having such structural and functional features as to allow to overcome the limitations and the drawbacks still limiting known methods, in particular capable of evaluating the mechanical and microstructural properties of the rolled metallic material in terms of traction yield strength and of a traction breaking strength, that method being suitable for application to all ferromagnetic and non-ferromagnetic metallic materials, in particular to austenitic and ferritic stainless steels, carbon steels, aluminum alloys, copper alloys, brass, etc. and being capable of continuously performing the necessary measurements during a production process.

### SUMMARY OF THE INVENTION

The solution idea underlying the present invention is to apply suitable combinations of deformation forces selected among compression forces, traction forces and bending moment on a section of the metallic material subjected to treatment with subsequent measurement of the lengthening undergone by the material itself, through an apparatus usable in a continuous treatment line of a metallic material, more particularly a steel or a metal alloy, the measurements being continuously performed both at low deformation speed, i.e. in a range comprised between 1*10⁻⁴ and 10*10⁻⁴ s⁻¹ corresponding to laboratory static conditions and at high deformation speed, i.e. in a range comprised between 0.1 and 10 s⁻¹ corresponding to dynamic conditions of a real production process, and then conveniently correlated to one another to evaluate the mechanical and microstructural properties of the material, in particular a steel.

Based on that solution idea the technical problem is solved by a method according to claim 1 and by an apparatus according to claim 11.

The features and the advantages of the method and of the evaluation apparatus according to the invention will be apparent from the following description, of embodiments thereof given by way of non-limiting examples with reference to the attached drawings.

### BRIEF DESCRIPTION OF THE DRAWINGS

In those drawings:

FIG. 1: schematically shows an evaluation apparatus of a rolled metallic material, suitable for implementing the method for continuously evaluating the mechanical and microstructural properties of that rolled metallic material according to the present invention;

FIG. 2: schematically shows means of application of deformation forces to a rolled metallic material in the shape of a Skin Pass comprising rolling rolls and a system of additional tensioning rolls of the evaluation apparatus of FIG. 1;

FIG. 3: schematically shows an alternative embodiment of means of application of deformation forces to a rolled metallic material in the shape of a tensive flattener of the apparatus of FIG. 1; and

FIG. 4: shows a diagram of correlation between values calculated with the evaluation apparatus of FIG. 1 at high and at low deformation speed.

### DETAILED DESCRIPTION

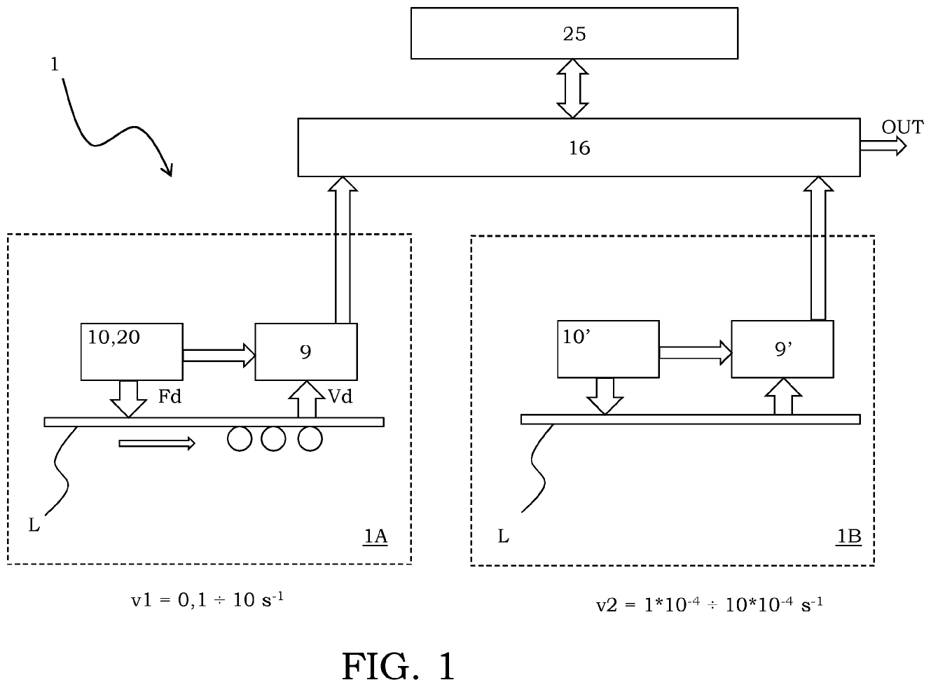

With reference to those figures, and more particularly to FIG. 1, an evaluation apparatus of a rolled metallic material L, such as a sheet metal or a strip made of that metallic material, in particular steel or a metal alloy, is globally indicated with 1. By way of non-limiting example, that evaluation apparatus 1 is usable in a continuous production line of carbon steels and reference will be made herebelow to that particular implementation by way of non-limiting example.

In particular, as it will be evident in the following description, the evaluation apparatus 1 allows the method for continuously evaluating mechanical and microstructural properties of the metallic material forming the rolled sheet L to be implemented, due to a measurement of the deformation of that material subjected to combinations of deformation forces selected among compression forces, traction forces and bending moment, and it is possible to insert it in a continuous industrial production process of such a metallic material, in particular of a steel, such as a continuous galvanizing, annealing, skin pass rolling line, etc.

More particularly, it will be pointed out how the evaluation apparatus 1 is capable of overcoming the limitation of the instruments available to date since it can perform measurements also for non-ferromagnetic metallic materials. Moreover, the evaluation apparatus 1, by implementing the proposed method, is capable of correlating measurements performed at low and high deformation speed, that low deformation speed being in a range comprised between 1*10⁻⁴ and 10*10⁻⁴ s⁻¹ and corresponding to laboratory static conditions and that high deformation speed being in a range comprised between 0.1 and 10 s⁻¹ and corresponding to dynamic conditions of a cold deformation process.

It is convenient to notice that the figures which represent schematic views of portions of the evaluation apparatus of the metallic material are not drawn to scale, but they are instead drawn in order to emphasize the important features of the invention.

Furthermore, in the figures, the different items are schematically represented, their shape being able to vary according to the desired application.

In its more general form, the present invention relates to a method for continuously evaluating mechanical and microstructural properties of a rolled metallic material L, in a cold deformation process, subjected to combinations of deformation forces selected among compression forces, traction forces and bending moment, applied at high deformation speed meaning a speed v1 in a range comprised between 0.1 and 10 s⁻¹ which corresponds to dynamic conditions and at low deformation speed meaning a speed v2 in a range comprised between 1*10⁻⁴ and 10*10⁻⁴ s⁻¹ which corresponds to laboratory static conditions.

The method comprises in particular the step of:
measuring characteristic parameters of the cold deformation process under dynamic conditions, comprising at least one value of temperature T, one deformation E and one deformation speed \acute{ε} of the rolled sheet L;

Conveniently according to the present invention the method further comprises the step of:
calculating the traction yield strength at high deformation speed σ_{YD} according to the following equation:

σ_{YD}=mσ_{c}+nσₜ+Pσ_{bend}

being:

σ_{c} a compression strength of the rolled sheet L when a compression force Fc is applied thereon;

σₜ a traction strength of the rolled sheet L when traction forces Tin and Tout are applied thereon;

σ_{bend} a strength due to the bending of the rolled sheet L when a bending moment is applied thereon; and

m, n, p are respectively a first, a second and a third parameter being a function of continuously-measured operating conditions of the cold deformation process and being a function of the rolled sheet L in terms of chemical composition and of preceding operating conditions of a hot deformation process, in terms of hot-deformation start and end temperature, winding temperature and grain size.

Advantageously, the method also comprises the steps of:
calculating the traction yield strength at low deformation speed σ_{YS} according to the following equation:

$${σ}_{Y s} = \frac{f}{α β} {σ}_{Y D}$$

being:

σ_{YD} the traction yield strength at high deformation speed;

f a statistical optimization factor between data measured at low deformation speed and at high deformation speed;

α a first characteristic parameter of the rolled sheet L being a function of the chemical composition of the rolled sheet L, of operating conditions of a hot deformation process of that rolled sheet L; and

β a second characteristic parameter of the rolled sheet L being a function of the cold deformation process calculated as:

$$β = \dot{ɛ} exp ( \frac{Q}{R T} )$$

being

\acute{ε} the deformation speed,

Q an activation energy of the deformation of that rolled sheet L evaluated through laboratory tests,

R the Boltzmann constant of ideal gases, and

T the temperature of the rolled sheet L.

It is pointed out that α and β are physical parameters.

Conveniently, the statistical optimization factor f has a value comprised between 0.1 and 1.5, the first characteristic parameter α has a value comprised between 0.05 and 5 and the second characteristic parameter β has a value comprised between 0.1 and 200.

Advantageously according to the present invention, the method also comprises the steps of:
calculating the compression strength ac of the rolled sheet L when a compression force Fc is applied thereon according to the following equation:

$${σ}_{c} = \frac{{F}_{c}}{W a}$$

being:

Fc such compression force applied to the rolled sheet L;

W the width of the rolled sheet L; and

a an arc formed by that rolled sheet L in correspondence of means of application of the compression force Fc;
calculating the traction strength σₜ of the rolled sheet L when traction forces Tin and Tout are applied thereon according to the following equation:

$${σ}_{t} = ( \frac{{T}_{i n} + {T}_{o u t}}{2} )$$

being:

Tin, Tout the traction forces in respective initial and final application positions, respectively indicated with in and out, to the rolled sheet L; and
calculating the bending strength σ_{bend} of the rolled sheet L when a bending moment is applied thereon according to the following equation:

$${σ}_{bend} = K ( \frac{Δ {P}_{bend}}{W s v} - Δ {T}_{bend} ) * \frac{1}{{A}_{bend}}$$

being:

K a parameter being function of a thickness s and of a friction coefficient μ of the rolled sheet L having values comprised between 0.1 and 10;

ΔP_{bend} a change in power of engines of means of application of the bending moment between respective initial and final application positions to the rolled sheet L, σP^{bend}=(Pₒᵤₜ−Pᵢₙ),

W the width of the rolled sheet L;

s the thickness of that rolled sheet L;

v a speed of the material L during the deformation process,

ΔT_{bend} a change in strength of the means of application of the bending moment between respective initial and final application positions to the rolled sheet L, ΔT_{bend}=(Tₒᵤₜ−Tᵢₙ), and

A_{bend} a lengthening of that rolled sheet L caused by the bending moment.

Conveniently, the method according to the present invention further comprises a step of calculating a traction breaking strength σ_{TS} of the rolled metallic material L according to the following equation:

σ_{TS}=σ_{YS}/Γ

being:

σ_{YS} the traction yield strength at low deformation speed, and

Γ a correlation factor, having a value comprised between 0.5 and 1. Also Γ is a physical parameter.

The method finally comprises a further step of calculating a recrystallization fraction Xrex of the rolled sheet L according to the following equation:

$${X}_{rex} = \frac{{σ}_{FH} - {σ}_{TS}}{{σ}_{FH} - {σ}_{RO}}$$

being:
σ_{FH} a traction breaking strength of the rolled sheet L after a cold deformation process, obtained under static conditions,

σ_{TS} the traction breaking strength under static conditions, and

σ_{RO} a traction breaking strength of the rolled sheet L with a completely recrystallized microstructure (Xrex=100%), obtained through laboratory tests.

As it will be explained herebelow, the values of the first, second and third parameter m, n and p depend on the type of means of application to the rolled sheet L of the deformation forces selected among compression forces, traction forces and bending moment. In particular, those first, second and third parameter m, n and p depend on process parameters such as forces applied to the rolled sheet L and subsequent lengthenings, the first parameter and the third parameter m and p also depend on the history of the material itself, as it will be explained herebelow.

In fact, the present invention also relates to an evaluation apparatus 1 capable of implementing a method for evaluating the mechanical and microstructural properties of the rolled metallic material L due to a correlation between those properties and the process parameters detected under high and low deformation speed conditions.

In its more general form, as schematically illustrated in FIG. 1, the evaluation apparatus 1 comprises at least:
first means of application, modulation and measurement 10, 20 of deformation forces selected among compression forces, traction forces and bending moment applied to the rolled sheet L during a deformation process at high deformation speed, in a range comprised between 0.1 and 10 s⁻¹ which corresponds to dynamic conditions; and
first means of measurement 9 of a deformation of the rolled sheet L following the application of the deformation forces at high deformation speed connected to the first means of application, modulation and measurement 10, 20.

Those first means of application, modulation and measurement 10, 20 of deformation forces and first means of measurement 9 of a deformation of the rolled sheet L following the application of the deformation forces at high deformation speed essentially form a process station 1A of the evaluation apparatus 1.

Such an evaluation apparatus 1 also comprises:
second means of application, modulation and measurement 10′ of deformation forces selected among compression forces, traction forces and bending moment applied to the rolled sheet L during a deformation process at low deformation speed in a range comprised between 1*10⁻⁴ and 10*10⁻⁴ s⁻¹ which corresponds to laboratory static conditions; and
second means of measurement 9′ of a deformation of the rolled sheet L following the application of the deformation forces at low deformation speed connected to the second means of application, modulation and measurement 10′.

Those second means of application, modulation and measurement 10′ of deformation forces and second means of measurement 9′ of a deformation of the rolled sheet L following the application of the deformation forces at low deformation speed essentially form a laboratory station 1B of the evaluation apparatus 1.

Conveniently, the evaluation apparatus 1 also comprises:
means of calculation 16 of the mechanical and microstructural properties of the rolled sheet L connected to the means of measurement 9, 9′ and suitable for implementing the above-described method; as well as
means of correlation 25 of data measured at high deformation speed and at low deformation speed.

More particularly, the means of application, modulation and measurement of deformation forces selected among compression forces, traction forces and bending moment operate so as to apply a compression force Fc comprised between 100 kN and 5000 kN and a traction force Ft comprised between 0.1 kN and 200 kN in order to obtain the desired deformation, in particular the controlled lengthening of the rolled metallic material L.

Conveniently, the lengthening of the rolled metallic material L is controlled so as to be comprised between 0.02%-30%, preferably comprised between 0.02%-5%.

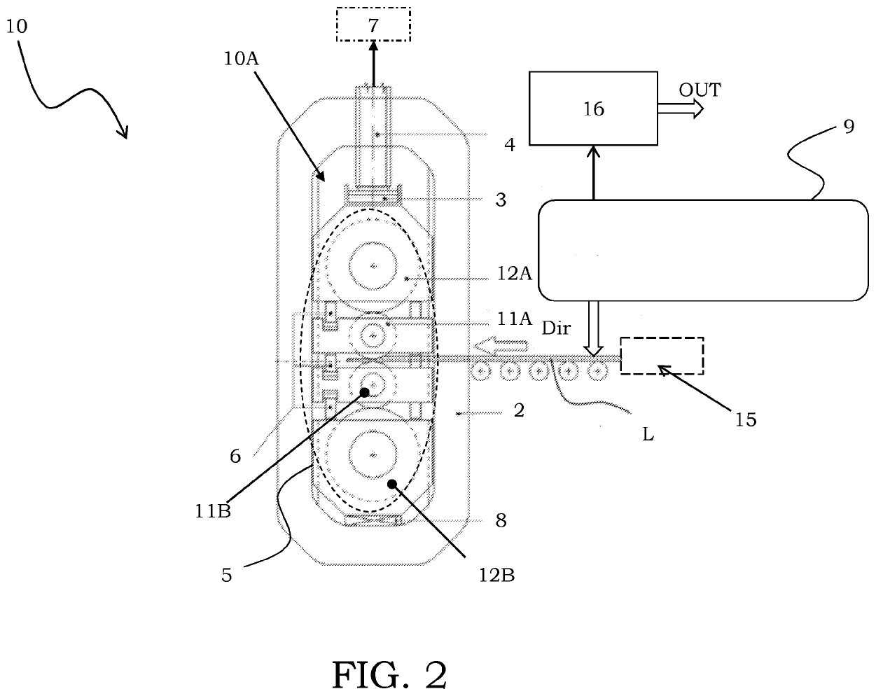

According to an embodiment, schematically illustrated in FIG. 2, the deformation forces are applied to the rolled sheet L through a deformation process performed through a cold rolling process with a Skin Pass 10. A Skin Pass 10 is a system essentially comprising at least rolling rolls suitable for applying to the rolled metallic material L convenient compression forces Fc and a system of additional tensioning rolls suitable for applying to that rolled sheet L convenient traction forces Tin, Tout in correspondence of input and output positions of those additional tensioning rolls.

More particularly, as illustrated in FIG. 2, the Skin Pass 10 comprises at least one block of tensioning rollers 10A, which the rolled sheet L whose mechanical and microstructural properties are to be measured is passed in, comprising at least one pair of work rolls, 11A, 11B suitable for receiving the rolled sheet L, preferably of high-resistance material, such as a steel HSS, or a steel with high chromium content, and in direct contact with the rolled sheet L, which an opposite compression force Fc and a pair of support rolls 12A, 12B are applied to suitable for giving a higher stiffness to the work rolls 11A, 11B and to the block of tensioning rollers 10A as a whole. More particularly, at least one support roll 12A, 12B, or shoulder rests on each of the work rolls 11A, 11B, typically having a higher diameter than the corresponding work roll 11A, 11B it presses on.

Each work roll 11A, 11B further comprises a respective central part, called table, whose surface is made particularly hard, in particular with hardness comprised in the range 30-80 HRC [Hardness Rockwell Cone] through convenient thermal treatments and respective ends whereon bearings are conveniently mounted, suitable for allowing the work rolls 11A, 11B to rotate, in particular capable of supporting high forces, like those involved in the rolled sheet production, in particular of steel, i.e. compression forces Fc in the range of 100 kN and 5000 kN and traction forces Ft in the range of 0.1 kN and 200 kN.

It can be in fact verified that the application of compression forces Fc comprised between 100 kN and 5000 kN and traction forces Ft comprised between 0.1 kN and 200 kN allow a deformation of the rolled metallic material L to be obtained, in particular a lengthening of the rolled sheet comprised between 0.02%-30%, preferably comprised between 0.02%-5%.

As shown in FIG. 2, the block of tensioning rollers 10A is inserted in a frame 2 of the Skin Pass 10 and is connected to at least one hydraulic roll 3, or HGC (Hydraulic Gauge Control) and to convenient adjustment bolts 4, only one being visible in FIG. 2, the distance between the work rolls 11A and 11B of the block of tensioning rollers 10A, usually indicated as roll gap, being mechanically fixed in an initial step through the adjustment bolts 4, placed on both sides of the block of tensioning rollers 10A and connected to a carter 5 which houses the support rolls 12A, 12B and the work rolls 11A, 11B and is equipped with convenient connecting hinges 6.

Only afterwards, the hydraulic roll 3 is used to accurately position those work rolls 11A, 11B of the block of tensioning rollers 10A, in particular in order to exert the desired compression force Fc for the rolled sheet L due to position sensors capable of controlling, through a control unit 7, servovalves for adjusting the positioning of the work rolls 11A, 11B.

In other words, the hydraulic roll 3, the adjustment bolts 4, the control unit 7 as well as convenient position sensors and servovalves form the means of application and modulation of the compression force Fc applied to the rolled sheet L of the Skin Pass 10.

Furthermore, the Skin Pass 10 comprises at least one load cell 8 capable of measuring the compression force Fc and conveniently connected to the control unit 7 so as to form the means of measurement of that compression force Fc.

Conveniently, the Skin Pass 10 further comprises a system of additional tensioning rolls suitable for applying to the rolled metallic material L convenient traction forces for example in the shape of a group of tensioning rollers 15, capable of applying convenient traction forces Tin, Tout to the rolled metallic material L through a double bridle system.

Conveniently, the group of tensioning rollers 15 comprises adjusting means (not shown) capable of varying the traction force Ft to be applied to the rolled sheet L, in the range between 0.1 kN and 200 kN; the value selected for that traction force Ft depends in particular on the format of the rolled sheet L, more particularly on the section thereof. Those adjusting means of the group of tensioning rollers 15 thus form the means of modulation of the traction force Ft applied to the rolled sheet L of the evaluation apparatus 1.

It can be verified that the overall effect of the traction forces Ft and compression forces Fc applied to the rolled sheet L results in a lengthening of the rolled sheet L itself, typically for a value comprised between 0.02%-30%, preferably comprised between 0.02%-5%.

The evaluation apparatus 1 further comprises means of calculation 16 connected to the means of measurement 9 and capable of evaluating the mechanical and microstructural properties of the rolled sheet L, provided as output OUT, implementing the above-described method.

The values obtained by the means of measurement 9 are provided to the means of calculation 16, which the values of the compression Fc and traction Ft forces applied to the rolled sheet L are also sent to, as obtained by the load cell 8.

It can be verified that, by using a Skin Pass 10 comprising rolling rolls suitable for applying to the rolled metallic material L a convenient compression force Fc and a system of additional tensioning rolls suitable for applying to that material convenient traction forces Tin, Tout in correspondence of input and output positions of those additional tensioning rolls, the first parameter m for the calculation of the traction yield strength at high deformation speed σ_{YD} is equal to:

$$m = \frac{a δ \dot{ɛ}}{{A}_{skp} * v ( 1 + \frac{μ a}{s} )}$$

being:

a the arc formed by the rolled sheet L in correspondence of the rolling rolls of the Skin Pass 10;

δ a first parameter which depends on features of the rolled sheet L, among which a chemical composition and operating conditions of a hot deformation process of that rolled sheet L;

\acute{ε} the deformation speed of the rolled sheet L during the rolling process with the Skin Pass 10,

Aₛₖₚ the lengthening of the rolled sheet L in correspondence of process conditions of the Skin Pass 10,

ρ the friction coefficient in correspondence of the rolling rolls of the Skin Pass 10,

μ and δ being physical parameters.

Moreover it occurs that, always in the case of the Skin Pass 10, the second parameter is equal to:

$$n = \frac{2}{\sqrt{3}}$$

while the third parameter p is null (p=0).

In other words, the traction yield strength at high deformation speed σ_{YD} is, in case of use of a Skin Pass 10, calculated by the following equation:

$${σ}_{YD} = \frac{a δ \dot{ɛ}}{{A}_{skp} * v ( 1 + \frac{μ a}{s} )} {σ}_{c} + \frac{2}{\sqrt{3}} {σ}_{t}$$

Conveniently, the contact arc a between the rolling rolls of the Skin Pass 10 and the rolled metallic material L is calculated according to the following equation:

$$a = \sqrt{R * {A}_{skp} * s * ( 1 + \frac{{F}_{c}}{W * s * C * {A}_{skp}} )}$$

being:

R the bending radius of the rolling rolls of the Skin Pass 10,

Aₛₖₚ the lengthening of the rolled sheet L in correspondence of the Skin Pass 10,

Fc the compression force applied by the Skin Pass 10, and

C a parameter which depends on a surface hardness of the rolling rolls of the Skin Pass 10, having a value comprised between 10000 and 200000, C being a physical parameter.

Furthermore, the first parameter δ has a value comprised between 0.5 and 1.5 and the friction coefficient μ has a value comprised between 0.001 and 0.5.

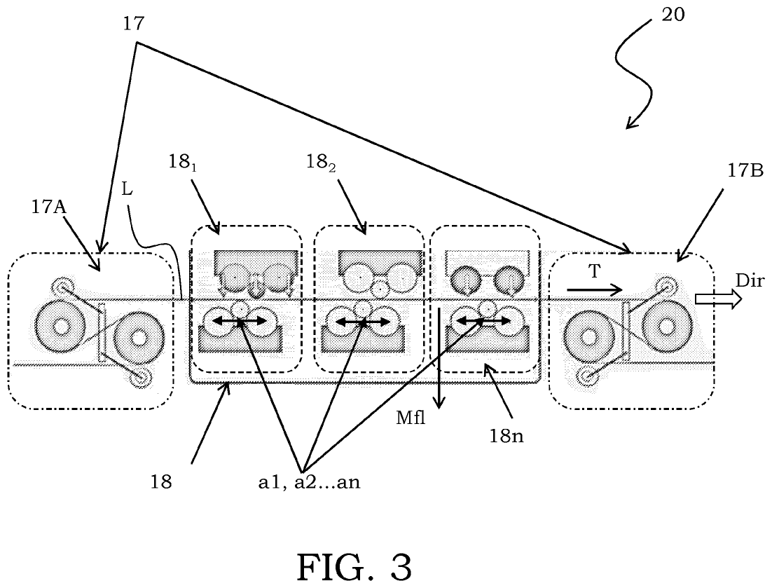

According to an alternative embodiment, schematically shown in FIG. 3, the deformation forces are applied to the rolled sheet L through a cold deformation process with a tensive flattener, essentially equipped with bending rolls capable of applying to that rolled sheet L a bending moment and with tensioning rolls associated to power engines capable of applying traction forces Tin, Tout in correspondence of input and output positions, together with a change in a power of the engines Pin, Pout.

More particularly, the tensive flattener 20 comprises at least one combined roll system, in particular made of high-resistance steel, suitable for applying convenient traction and compression forces to the rolled sheet L. More particularly, the deformation to the rolled sheet L is given by the joint action of a longitudinal strength or pull T with respect to a displacement direction Dir of the rolled sheet L, obtained through tensioning rollers 17, and by a bending moment Mfl generated by a series of curving rolls 18.

As shown in FIG. 3, in a preferred embodiment, the tensive flattener 20 comprises at least first and second tensioning rollers 17A and 17B positioned respectively upstream and downstream of the curving rolls 18, with reference to the displacement direction Dir.

Furthermore, the tensive flattener 20 comprises a plurality of groups of curving rolls, indicated as 18₁, 18₂ and 18n, cascade-connected to one another.

Conveniently, the tensive flattener 20 applies a deformation force on the rolled sheet L comprising a traction or pull component Tin, Tout generated by the tensioning rollers 17 and by a bending moment Mfl generated by the bending imposed to the rolled sheet L when it passes through the curving rolls 18, whose interference generates a controlled bending of the rolled sheet itself.

The overall deformation undergone by the rolled metallic material L is typically lower than 10% and it is measured through the means of measurement of the deformation of the rolled sheet L, such as the above-indicated means of measurement 9.

It can be verified that, by using a tensive flattener 20 comprising bending rolls capable of applying to the rolled metallic material L the bending moment and tensioning rolls associated to power engines capable of applying the traction forces Tin, Tout, the first and the second parameter m, n for the calculation of the traction yield strength at high deformation speed σ_{YD} are null (m=0, n=0) while the third parameter p is equal to:

$$p = τ ( \frac{s}{( {ρ}_{eq} + s / 2 ) {A}_{sp}} )$$

being:

τ a third characteristic parameter of the rolled sheet L being a function of chemical composition and operating conditions of a hot deformation process of that rolled sheet L, τ being a physical parameter with values comprised between 0.1 and 10.

ρ_{eq} the equivalent bending radius of the rolled sheet L in correspondence of the tensive flattener 20, and

Aₛₚ the lengthening of the rolled sheet L in correspondence of the tensive flattener 20.

The traction yield strength at high deformation speed σ_{YD}, in case of use of a tensive flattener 20, is thus calculated by the following equation:

$${σ}_{YD} = τ ( \frac{s}{( {ρ}_{eq} + s / 2 ) {A}_{sp}} ) * {σ}_{bend}$$

It is also possible to use a Skin Pass 10, in combination with a tensive flattener 20 to apply to the rolled sheet L compression, traction and bending-moment forces, the parameters of the equation for the calculation of the traction yield strength at high deformation speed σ_{YD} being in that case:

$$m = \frac{a δ \dot{ɛ}}{{A}_{skp} * v ( 1 + \frac{μ a}{s} )}$$

$$n = \frac{2}{\sqrt{3}} , and$$

$$p = τ ( \frac{s}{( {ρ}_{eq} + s / 2 ) {A}_{sp}} )$$

The means of application, modulation and measurement 10′ of the deformation forces to the rolled sheet as well as the means of measurement 9′ at low deformation speed in correspondence of the laboratory station 1B are made by a traction machine.

The Applicant performed a verification of the proposed evaluation method for each class of metallic materials of interest, such as for example AHSS, interstitial free, stainless steels, aluminum alloys, etc., and he could ascertain that the values calculated by the above-described method and the experimental data differ by less than 1%.

For the validation of the suggested method and apparatus and in particular for the evaluation of the new selected physical parameters a long experimental activity was carried out through a high series of traction tests, of chemical analyses and of microstructural examinations, and a related numerical analysis was performed so as to be able to correctly compare and correlate data obtained at low deformation speed with data obtained ad high deformation speed, so as to evaluate through the present invention the real mechanical and microstructural properties of the material.

The results of those verifications are mentioned in the following examples.

### Example 1

The measurements were performed according to the above-suggested method in a continuous galvanizing line to a strip of steel grade S320GD (EN10346).

The measurement of the mechanical features was continuously performed through a skin pass characterized by the following operating conditions:

| Skin pass process parameters | Unit of measure | Value |
|---|---|---|
| Fc Skin Pass Force | KN | 2030 |
| A_{SKP} Skin Pass lengthening | % | 0.006 |
| Tᵢₙ Skin Pass input strength | KN | 160 |
| Tₒᵤₜ Skin Pass output strength | KN | 170 |
| R Roll radius | Mm | 190 |
| Parameter C (roll hardness) | — | 40000 |
| T Strip temperature | ° C. | 80 |
| μ friction coefficient | — | 0.02 |
| a contact arc | mm | 2.8 |
| W strip width | mm | 1500 |
| s strip thickness | mm | 2.4 |
| \dot{ε} deformation speed | s⁻¹ | 2.9 |
| V strip speed | m/s | 80 |

The parameters relating to the material of the present example are instead mentioned in the following table:

| Material parameters | Unit of measure | Value |
|---|---|---|
| f | — | 1.12 |
| δ | — | 0.89 |
| α | s | 0.16 |
| β | s⁻¹ | 9.16 |
| R_{FH} | Mpa | 650 |
| Ro | MPa | 360 |
| Q | J/mol | 1500 |
| Γ | — | 0.84 |

α, β, μ, C, Γ and δ being physical parameters.

The calculation of the mechanical features (yield, breaking resistance) and of the recrystallization fraction is performed through the following step.

The yield strength σ_{YD} at high deformation speed is calculated according to the general equation:

σ_{YD}=mσ_{c}+nσₜ+pσ_{bend}

In the case of the skin pass it gets that p=0,

$$n = = \frac{2}{\sqrt{3}} ,$$

m is calculated by the equation

$$m = \frac{a δ \dot{ɛ}}{{A}_{skp} * v ( 1 + \frac{μ a}{s} )}$$

Whereby the yield strength of the material of the present example under high deformation speed conditions is:

σ_{YD}=438 MPa

The mechanical features of a metallic material evaluated through traction tests are measured in an almost static state i.e. at low deformation speed (range 10⁻³ s⁻¹÷10⁻⁴ s⁻¹).

From the performance of a very wide series of laboratory tests an empirical relation of this type was established between the yield strength at low and high deformation speed:

$${σ}_{Ys} = \frac{f}{αβ} {σ}_{YD}$$

Based on the above-mentioned parameters the yield value of the material being concerned (S320GD) can be calculated

σ_{Ys}=348 MPa

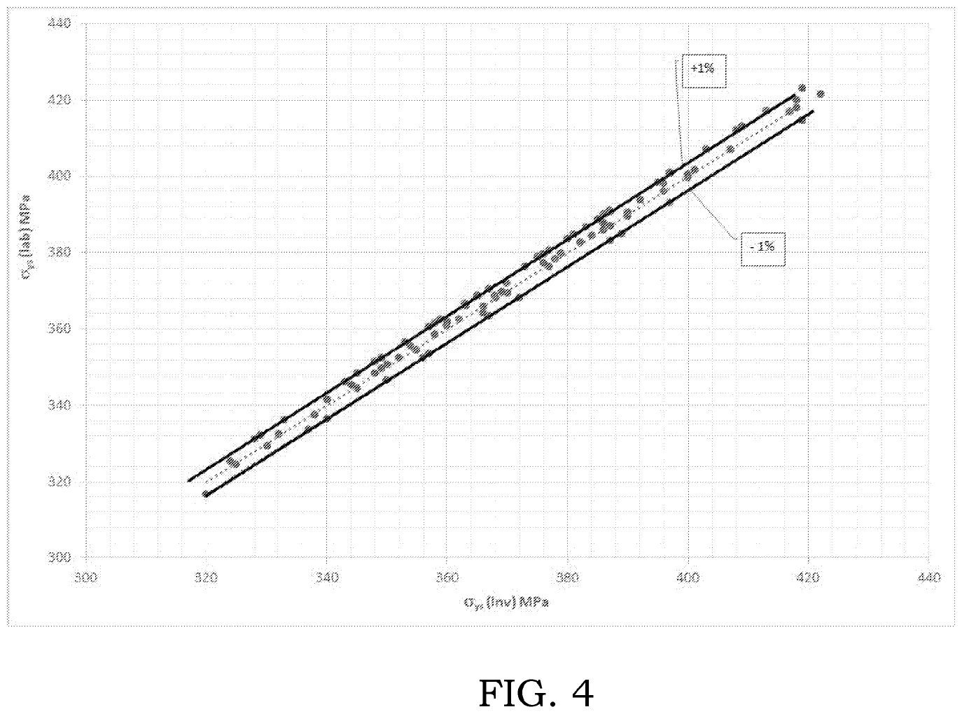

The values calculated through the apparatus provided in the present patent application on the basis of the experimental values obtained through laboratory traction tests are mentioned in the diagram in FIG. 4. As it can be noticed the concordance is excellent since the values calculated according to the suggested method have a very limited dispersion (<±1%) with respect to experimental values.

The breaking strength σ_{TS} of the material is evaluated by an equation

σ_{TS}=σ_{YS}/Γ

In this example the calculated breaking strength value is σ_{TS}=574 Mpa.

The calculation of the recrystallized fraction is performed by the following empirical equation:

$${X}_{rex} = \frac{{σ}_{FH} - {σ}_{TS}}{{σ}_{FH} - {σ}_{RO}}$$

where σ_{FH} is the traction breaking strength of the material after cold rolling, σ_{TS} is the traction breaking strength continuously calculated in the preceding step, and σ_{RO} is the traction breaking strength of the material under complete recrystallization conditions (Xrex=100%). In the case of the steel grade S320GD the parameters σ_{FH} and σ_{RO} were evaluated through laboratory tests and appeared to be: 650 MPa and 360 MPa whereby in this example it appears Xrex=100%.

### Example 2

The measurements were performed according to the above-suggested method in a continuous galvanizing line to a strip of steel grade HX420LAD (EN10346).

The measurement of the mechanical features is continuously performed by a tensive flattener characterized by the following operating conditions:

| Flattener process parameters | Unit of measure | Value |
|---|---|---|
| A_{SP} flattener lengthening | — | 0.006 |
| Tᵢₙ flattener input strength | KN | 100 |
| Tₒᵤₜ flattener output strength | KN | 140 |
| ΔP_{bend} (Pₒᵤₜ − Pᵢₙ) | MPa * s/mm | 15 |
| ρ_{eq} equivalent radius of the | Mm | 60 |
| flattener rolls | | |
| T strip temperature | ° C. | 50 |
| μ friction coefficient | — | 0.1 |
| Parameter K | — | 0.24 |
| W strip width | mm | 1250 |
| s strip thickness | mm | 2.4 |
| \dot{ε} deformation speed | s⁻¹ | 5.1 |
| v strip speed | m/s | 120 |

The parameters relating to the material of the present example are instead mentioned in the following table.

| Material parameters | Unit of measure | Value |
|---|---|---|
| f | — | 1.02 |
| τ | — | 0.89 |
| α | s | 0.25 |
| β | s⁻1 | 5.01 |
| σ_{FH} | Mpa | 790 |
| σ_{RO} | MPa | 450 |
| Q | J/mol | 1860 |
| Γ | — | 0.74 |

α, β, μ, C, Γ and τ being physical parameters

The calculation of the mechanical features (yield, breaking resistance) and of the recrystallization fraction is performed through the following steps.

The yield strength σ_{YD} at high deformation speed is calculated according to the general equation:

σ_{YD}=mσ_{c}+nσₜ+pσ_{bend}

Having used a tensive flattener, it gets m=0, n=0 and thus

σ_{YD}=pσ_{bend}

where the parameter p is calculated by the formula:

$$p = τ ( \frac{s}{( {ρ}_{eq} + s / 2 ) {A}_{sp}} )$$

τ being another parameter connected to the chemical composition and hot rolling process.

The strength due to the strip bending is thus calculated by the following equation:

$${σ}_{bend} = K ( \frac{Δ {P}_{bend}}{W s v} - Δ {T}_{bend} ) * \frac{1}{{A}_{bend}}$$

By introducing the values of the process and material parameters it obtains

σ_{bend}=90 MPa

The dynamic traction yield becomes therefore:

$${σ}_{YD} = τ ( \frac{s}{( {ρ}_{eq} + s / 2 ) {A}_{sp}} ) * {σ}_{bend}$$

In the case of this example it gets σ_{YD}=540 MPa.

The mechanical features of a metallic material evaluated through traction tests are measured in an almost static state i.e. at low deformation speed (range 10⁻³ s⁻¹÷10⁻⁴ s⁻¹).

From the performance of a very wide series of laboratory tests an empirical relation of this type was established between the yield strength at low and high deformation speed:

$${σ}_{Y s} = \frac{f}{α β} {σ}_{Y D}$$

Based on the above-mentioned parameters the yield value of the material being concerned (HX420LAD) can be calculated

σ_{Ys}=432 MPa

The material breaking strength is evaluated by an equation

σ_{TS}=σ_{YS}/Γ

In this example the calculated breaking strength value is σ_{TS}=584 Mpa.

The calculation of the recrystallized fraction is performed by the following empirical equation:

$${X}_{rex} = \frac{{σ}_{F H} - {σ}_{T S}}{{σ}_{F H} - {σ}_{R O}}$$

where σ_{FH} is the traction breaking strength of the material after cold rolling, σ_{TS} is the traction breaking strength continuously calculated in the preceding step, and σ_{RO} is the traction breaking strength of the material when in complete recrystallization conditions (Xrex=100%). In the case of the steel grade HX420LAD it appears Xrex=100%.

In conclusion, the evaluation apparatus according to the invention allows a method for evaluating the mechanical and microstructural properties of a rolled metallic material, out of steel or metal alloys, to be implemented, usable on the continuous treatment lines of alloyed and unalloyed steels and in general of metal alloys.

More particularly, that method provides the application on a rolled sheet of convenient combinations of deformation forces selected among compression forces, traction forces and bending moment and following measurements at low deformation speed (between 1*10⁻⁴ and 10*10⁻⁴ s⁻¹ corresponding to laboratory static conditions) and at high deformation speed (between 0.1 and 10 s⁻¹ corresponding to dynamic process conditions) so as to calculate the mechanical and microstructural properties of the rolled sheet itself, in particular in terms of traction yield strength at low deformation speed σ_{YS}, traction yield strength at high deformation speed σ_{YD} and traction breaking strength σ_{TS}.

It is underlined that the method and the evaluation apparatus are usable for all ferromagnetic and non-ferromagnetic metallic materials, in particular austenitic and ferritic stainless steels, carbon steels, aluminum alloys, copper alloys, etc.

Moreover, advantageously according to the present invention, the proposed method allows also the percentage of recrystallization of a cold-deformed rolled sheet to be evaluated, for example after a high-temperature annealing process.

Obviously, in order to meet contingent and specific requirements, a person skilled in the art will be allowed to bring several modifications and alternatives to the above-described evaluation apparatus and method, all comprised in the scope of protection of the invention as defined by the following claims.

1. A method for continuously evaluating mechanical and microstructural properties of a rolled metallic material in a cold deformation process, wherein a rolled sheet (L) is subjected to combinations of deformation forces selected among compression forces, tensile forces and bending moment,
wherein said deformation forces are applied at low deformation rate in a range comprised between 1*10⁻⁴ and 10*10⁻⁴ s⁻¹ which corresponds to static conditions,
and wherein said deformation forces are applied at high deformation rate in a range comprised between 0.1 and 10 s⁻¹ which corresponds to dynamic conditions,
said method comprising the step of:
measuring characteristic parameters of said cold deformation process under dynamic conditions, said characteristic parameters comprising temperature (T), deformation (ε) and deformation rate (\acute{ε}) of said rolled sheet (L);
measuring said deformation forces selected among compression forces (Fc), tensile forces (Tin, Tout) and bending moment applied to the rolled sheet (L) at high deformation rate;
Hi calculating a tensile yield strength at high deformation rate (σ_{YD}) according to the following equation:
σ_{YD}=mσ_{c}+nσₜ+pσ_{bend}
being:
σ_{c} a compression stress of said rolled sheet (L) when said compression force (Fc) is applied thereon;
σₜ a tensile stress of said rolled sheet (L) when said tensile forces (Tin, Tout) are applied thereon;
σ_{bend} a stress due to the bending of said rolled sheet (L) when said bending moment is applied thereon; and
m, n, p are a first, a second and a third parameter respectively, said first, said second and said third parameter being a function of continuously-measured operating conditions of said cold deformation process and further being a function of said rolled sheet (L) in terms of chemical composition and further being a function of preceding operating conditions of a hot deformation process of said rolled sheet (L), in terms of hot-deformation start and end temperature, winding temperature and grain size;
calculating a tensile yield strength at low deformation rate (σ_{YS}) as a function of said calculated tensile yield strength at high deformation rate (σ_{YD}), according to the following equation:
$${σ}_{Y s} = \frac{f}{α β} {σ}_{Y D}$$
being:
σ_{YD} said tensile yield strength at high deformation rate;
f a statistical optimization factor between data measured at low deformation rate and at high deformation rate;
α a first characteristic parameter of said rolled sheet (L) being a function of chemical composition of said rolled sheet (L) and of operating conditions of a hot deformation process of said rolled sheet (L); and
β a second characteristic parameter of said rolled sheet (L) being a function of said cold deformation process, said second characteristic parameter being calculated as:
$$β = \dot{ɛ} exp ( \frac{Q}{R T} )$$
being
\acute{ε} said deformation rate,
Q an activation energy of said deformation of said rolled sheet (L) evaluated through laboratory tests,
R the Boltzmann constant of ideal gases, and
T said temperature of said rolled sheet (L).

2. The method for evaluating mechanical and microstructural properties of a rolled metallic material (L) according to claim 1, wherein further comprising the following steps of:
calculating said compression stress (σ_{c}) of said rolled sheet (L) when a compression force (Fc) is applied thereon according to the following equation:
$${σ}_{c} = \frac{{F}_{c}}{W a}$$
being:
Fc said compression force applied to said rolled sheet (L);
W a width of said rolled sheet (L); and
a an arch formed by said rolled sheet (L) in correspondence of means of application of said compression force (Fc);
calculating said tensile stress (σₜ) of said rolled sheet (L) when tensile forces (Tin, Tout) are applied thereon according to the following equation:
$${σ}_{t} = ( \frac{{T}_{i n} + {T}_{out}}{2} )$$
being:
Tin, Tout said tensile forces in respective initial and final application positions (in, out) to said rolled sheet (L); and
calculating said bending stress (σ_{bend}) of said rolled sheet (L) when a bending moment is applied thereon according to the following equation:
$${σ}_{bend} = K ( \frac{Δ {P}_{bend}}{W s v} - Δ {T}_{bend} ) * \frac{1}{{A}_{bend}}$$
being:
K a parameter being a function of a thickness (s) and of a friction coefficient (μ) of said rolled sheet (L);
ΔP_{bend} a change in power of motors of means of application of said bending moment between respective initial and final application positions (in, out) to said rolled sheet (L), ΔP_{bend}=(Pₒᵤₜ−Pᵢₙ),
W said width of said rolled sheet (L);
s said thickness of said rolled sheet (L),
v a speed of said rolled sheet (L) during said deformation process,
ΔT_{bend} a change in tension of said means of application of said bending moment between respective initial and final application positions (in, out) to said rolled sheet (L), ΔT_{bend}=(Tₒᵤₜ−Tᵢₙ), and
A_{bend} a lengthening of said rolled sheet (L) caused by said bending moment.

3. The method for evaluating mechanical and microstructural properties of a rolled metallic material (L) according to claim 2, wherein said deformation forces are applied to said rolled sheet (L) through a deformation process performed through a cold rolling process with a Skin Pass comprising rolling rolls suitable for applying to said rolled sheet (L) said compression force (Fc) and a system of additional tensioning rolls suitable for applying to said rolled sheet (L) said tensile forces (Tin, Tout) in correspondence of input and output positions (in, out) of said additional tensioning rolls, wherein said first, second and third parameter (m, n, p) are equal to:
$$m = \frac{a δ \dot{ɛ}}{{A}_{skp} * v ( 1 + \frac{μ a}{s} )}$$
being:
a said arc formed by said rolled sheet (L) in correspondence of said rolling rolls of said Skin Pass;
δ a first parameter which depends on features of said rolled sheet (L), among which a chemical composition and operating conditions of a hot deformation process of said rolled sheet (L);
\acute{ε} said deformation rate of said rolled sheet (L) during said rolling process with Skin Pass,
Aₛₖₚ a lengthening of said rolled sheet (L) in correspondence of process conditions of said Skin Pass,
μ said friction coefficient in correspondence of said rolling rolls of said Skin Pass, and
$$n = \frac{2}{\sqrt{3}} , and p = 0$$
said tensile yield strength at high deformation rate (σ_{YD}) being thus calculated by the following equation:
$${σ}_{YD} = \frac{a δ \dot{ɛ}}{{A}_{skp} * v ( 1 + \frac{μ a}{s} )} {σ}_{c} + \frac{2}{\sqrt{3}} {σ}_{t}$$

4. The method for evaluating mechanical and microstructural properties of a rolled sheet (L) according to claim 3, wherein said contact arc (a) is calculated according to the following equation:
$$a = \sqrt{R * {A}_{skp} * s * ( 1 + \frac{{F}_{c}}{W * s * C * {A}_{skp}} )}$$
being:
R a bending radius of said rolling rolls of said Skin Pass,
Aₛₖₚ said lengthening of said rolled sheet (L) in correspondence of said Skin Pass,
Fc said compression force applied by said Skin Pass, and
C a parameter which depends on a surface hardness of said rolling rolls of said Skin Pass, having a value comprised between 10000 and 200000.

5. The method for evaluating mechanical and microstructural properties of a rolled metallic material (L) according to claim 2, wherein said deformation forces are applied to said rolled sheet (L) through a cold deformation process with a tension leveler equipped with bending rolls capable of applying to said rolled sheet (L) said bending moment and tensioning rolls associated to power motors capable of applying said tensile forces (Tin, Tout) in correspondence of input and output positions (in, out) through said tensioning rolls and to a change in power of said motors (Pin, Pout), and in that the first, second and third parameter (m, n, p) are equal to:
$$m = 0 , \\ n = 0 , and$$
$$p = τ ( \frac{s}{( {ρ}_{e q} + s / 2 ) {A}_{s p}} )$$
being:
τ a third characteristic parameter of said rolled sheet (L) being a function of chemical composition and operating conditions of a hot deformation process of said rolled sheet (L),
ρ_{eq} an equivalent bending radius of said rolled sheet (L) in correspondence of said tension leveler, and
Aₛₚ a lengthening of said rolled sheet (L) in correspondence of said tension leveler,
said tensile yield strength at high deformation rate (σ_{YD}) being thus calculated by the following equation:
$${σ}_{Y D} = τ ( \frac{s}{( {ρ}_{e q} + s / 2 ) {A}_{s p}} ) * {σ}_{bend}$$

6. The method for evaluating mechanical and microstructural properties of a rolled metallic material (L) according to claim 3, wherein said deformation forces are applied to said rolled sheet (L) through a cold deformation process with a Skin Pass in combination with a tension leveler and in that said first, second and third parameter (m, n, p) are equal to:
$$m = \frac{a δ \dot{ɛ}}{{A}_{s k p} * v ( 1 + \frac{μ a}{s} )} \\ n = \frac{2}{\sqrt{3}} , and$$
$$p = τ ( \frac{s}{( {ρ}_{e q} + s / 2 ) {A}_{s p}} )$$

7. The method for evaluating mechanical and microstructural properties of a rolled metallic material (L) according to claim 1, further comprising a step of calculating a ultimate tensile strength (σ_{TS}) of said rolled sheet (L) according to the following equation:
σ_{TS}=σ_{YS}/Γ
being:
σ_{YS} said tensile yield strength at low deformation rate, and
Γ a correlation factor, having a value comprised between 0.5 and 1.

8. The method for evaluating mechanical and microstructural properties of a rolled metallic material (L) according to claim 7, further comprising a further step of calculating a recrystallization fraction (Xrex) of said rolled sheet (L) according to the following equation:
$${X}_{r e x} = \frac{{σ}_{F H} - {σ}_{T S}}{{σ}_{F H} - {σ}_{R O}}$$
being:
σ_{FH} an ultimate tensile strength of said rolled sheet (L) after a cold deformation process, obtained under static conditions,
σ_{TS} said ultimate tensile strength under static conditions, and
σ_{RO} an ultimate tensile strength of said rolled sheet (L) with a completely recrystallized microstructure (Xrex=100%), obtained through laboratory tests.

9. The method for evaluating mechanical and microstructural properties of a rolled metallic material (L) according to claim 1, wherein said statistical optimization factor (f) has a value comprised between 0.1 and 1.5, said first characteristic parameter (α) has a value comprised between 0.05 and 5 and said second characteristic parameter (β) has a value comprised between 0.1 and 200.

10. The method for evaluating mechanical and microstructural properties of a rolled metallic material (L) according to claim 3, wherein said first parameter (δ) has a value comprised between 0.5 and 1.5 and said friction coefficient (μ) has a value comprised between 0.001 and 0.5, said parameter (K) has a value comprised between 0.1 and 10 e said parameter (τ) has a value comprised between 0.1 and 10.

11. An evaluation apparatus of mechanical and microstructural properties of a rolled metallic material in a cold deformation process, comprising:
first means of application, modulation and measurement of deformation forces selected among compression forces (Fc), tensile forces (Tin, Tout) and bending moment applied to a rolled sheet (L) during a deformation process at high deformation rate in a range comprised between 0.1 and 10 s⁻¹ which corresponds to dynamic conditions;
first means of measurement of a deformation of said rolled sheet (L) following said application of said deformation forces at high deformation rate connected to said first means of application, modulation and measurement;
second means of application, modulation and measurement of deformation forces selected among compression forces (Fc), tensile forces (Tin, Tout) and bending moment applied to said rolled sheet (L) during a deformation process at low deformation rate in a range comprised between 1*10⁻⁴ and 10*10⁻⁴ s⁻¹ which corresponds to static conditions;
second means of measurement of a deformation of said rolled sheet (L) following said application of said deformation forces at low deformation rate connected to said second means of application, modulation and measurement;
means of calculation of said mechanical and microstructural properties of said rolled sheet (L) connected to said first and second means of measurement and adapted for implementing a method for continuously evaluating mechanical and microstructural properties of a rolled metallic material according to claim 1; and
means of correlation of data measured at high deformation rate and at low deformation rate.

12. The evaluation apparatus according to claim 11, wherein said first means of application, modulation and measurement of deformation forces are selected between: a Skin Pass comprising rolling rolls suitable for applying to said rolled sheet (L) a compression force (Fc) and a system of additional tensioning rolls suitable for applying to said rolled sheet (L) tensile forces (Tin, Tout) in correspondence of input and output positions (in, out) of said additional tensioning rolls; or a tension leveler equipped with bending rolls capable of applying to said rolled metallic material (L) said bending moment and with tensioning rolls associated to power motors capable of applying said tensile forces and/or a combination thereof.

13. The evaluation apparatus according to claim 11, wherein said first means of application, modulation and measurement of deformation forces are suitable for applying to said rolled sheet (L) traction forces (Ft) comprised between 0.1 kN and 200 kN, compression forces (Fc) comprised between 100 kN and 5000 kN or a bending moment so as to obtain a deformation, in particular a lengthening of said rolled sheet (L) comprised between 0.02%-30%, preferably comprised between 0.02%-5%.
